(19) United States
(12) Patent Application Publication
(10) Pub. No.: US 2026/0000799 A1
(43) Pub. Date: Jan. 1, 2026

(54) LOW-TEMPERATURE STERILIZATION DEVICE AND METHOD THEREOF

(71) Applicant: Lishing Lambert Ding, Taipei (TW)

(72) Inventor: Lishing Lambert Ding, Taipei (TW)

(21) Appl. No.: 18/758,292

(22) Filed: Jun. 28, 2024

(51) Int. Cl. A61L 2/10; A61L 2/08; A61L 2/20; A61L 2/24; A61L 2/26

(52) U.S. Cl. CPC A61L 2/10; A61L 2/088; A61L 2/202; A61L 2/24; A61L 2/26; A61L 2202/122; A61L 2202/24

(57) ABSTRACT

A low-temperature sterilization device and a method thereof, which are used to decontaminate various medical equipment. The low-temperature sterilization device is designed to automatically clean, disinfect and sterilize contaminated medical equipment by applying ultrasonic waves, ultraviolet rays and ozone, along with super oxidant or sterilant in aqueous solutions to achieve rapid and high quality cleaning, desorption, destruction and decontamination reactions to remove all adhering pollutants, microorganisms, bacteria and viruses from all crevices, pores and corners of contaminated medical equipment, thus, to ensuring sterilization for medical equipment especially for those do not sustain high temperature sterilization.

### CROSS-REFERENCE TO RELATED APPLICATION

### Background

### Technical Field

The current application relates to a low-temperature sterilization device and method, particularly referring to a low-temperature sterilization device and method using ultrasonic waves, chemical agents, ozone, ultraviolet light, and photocatalysts to enhance the disinfection and sterilization effect of medical equipment.

### Description of Related Art

In today's healthcare environment, the use of invasive catheters and treatment procedures has become a common medical practice, but it has also raised considerable concerns about healthcare-associated cross infections.

One of the most common and effective methods for medical equipment sterilization in medical facilities is high-temperature steam sterilization, also known as autoclaving or autoclave sterilization. This method utilizes high temperature and pressure to effectively eliminate bacteria, viruses, and other microorganisms from the surface of medical equipment using steam. It is a widely used sterilization method applicable to various types of medical equipment, including surgical instruments, instrument containers, and metallic products.

In addition, chemical sterilization methods are also common choices, such as using hydrogen peroxide plasma, or ethylene oxide gas for sterilization.

However, none of the above methods are suitable for endoscopes because of their unique structure and material composition, making traditional high-temperature steam sterilization and chemical sterilization methods difficult to ensure effective disinfection and sterilization. Therefore, specialized disinfection and sterilization methods need to be developed for special equipment like endoscopes to ensure their safe and hygienic use.

Currently, the sterilization method for special equipment like endoscopes is ethylene oxide gas sterilization. Although this method meets the standards for low-temperature sterilization of special equipment like endoscopes, it brings about a series of inconveniences. Firstly, ethylene oxide, the substance used in this method, is a carcinogen, posing significant health risks to human and the environment.

Furthermore, due to the lack of an automatic cleaning function, users need to perform additional cleaning steps before endoscopes sterilization, increasing the complexity of labor and time costs. Additionally, this method requires minimum aeration parameters with 12 hours in an aeration chamber at 50-57° C. (122-135° F.) or 7 days at room temperature making the equipment unavailable for immediate reuse. For expensive endoscopic medical devices, the cost of standby equipment is extremely high. Therefore, this disinfection/sterilization method not only affects the rapid reuse of special equipment like endoscopes but also brings additional challenges and cost burdens to users and healthcare facilities.

The applicant has filed a Chinese invention patent CN1961964A, presenting a low-temperature disinfection and sterilization method along with its apparatus. This method primarily targets the disinfection, sterilization, and decontamination of contaminated reusable medical equipment. The process involves rapid detachment, destruction, and decomposition reactions using ultrasound, ultraviolet light, ozone, and disinfectants in aqueous solutions. This allows for deep and thorough cleaning, disinfection, and sterilization of various contaminated medical equipment, ensuring complete removal and eradication of contaminants, bacteria, and viruses. Furthermore, the applicant has made further improvements by proposing an innovative low-temperature sterilization device. This device adjusts the concentration of the aqueous solution through measurement and metering units to ensure the disinfectant concentration within a certain range. Additionally, it can automatically perform cleaning using ultrasound, enhancing sterilization stability and reliability. The invention can accomplish automatic cleaning and disinfection/sterilization of contaminated reusable medical equipment at room temperature in approximately 30 minutes, significantly reducing the labor and time required for cleaning such equipment.

WO2023246951A1 describes a helmet drying, sterilizing, and disinfecting machine capable of automatically detecting the dryness level thereof, which comprises a housing and a base; a placement surface used for placing a helmet is formed at the top of the housing, and raised helmet supports are arranged on the placement surface; a PCBA board, a drying assembly, and a sterilization and disinfection assembly are mounted within the housing, the drying assembly and the sterilization and disinfection assembly are each provided with a protection system, and the helmet placement surface is provided with a wetness level sensor.

WO2024051459A1 describes an efficient, bacteriostatic, minimally invasive collection device for a great saphenous vein, which comprises a grip, a sleeve cutter, and a protection sleeve. The sleeve cutter can be disinfected by means of an ultraviolet lamp tube in the bacteriostatic cylinder, so that an efficient bacteriostatic effect is achieved.

### SUMMARY

In one aspect, the present invention is directed to a low-temperature sterilization device and method.

The low-temperature sterilization device is provided. The low-temperature sterilization device utilizes slightly acidic electrolyzed water to disinfect and sterilize contaminated medical equipment placed within the device, effectively removing adhered contaminants, bacteria and virus from all crevices and corners of the contaminated medical equipment, wherein the low-temperature sterilization device comprises a reaction tank, an operating device, an ultrasonic device, a metering unit, a feeding pump, and a recirculation pump.

The reaction tank is arranged to accommodate the contaminated medical equipment for disinfection and sterilization processing. The operating unit is positioned above the reaction tank, providing users with the options to select one of a plurality of automated running programs to control and monitor the entire disinfection and sterilization process. The ultrasonic device is utilized to detach the adhered contaminants, bacteria and virus from the contaminated medical equipment through ultrasonic vibrations with the frequency ranges from 20 hertz to 1 giga hertz. The metering unit is used to calculate the required dosage of the chemical agents based on the volume of the aqueous solution in the reaction tank to provide a concentration of the aqueous solution within a range of 100 ppm to 10000 ppm. The feeding pump is installed within the operating unit, importing the chemical agents from the chemical tank into the reaction tank according to the required dosage. The recirculation pump connects respectively to the channels of medical equipment and the reaction tank, for delivering a required dosage of chemical agents into the contaminated medical equipment channels.

According to another embodiment, the low-temperature sterilization device further comprises an ultraviolet (UV) light device. The ultraviolet light device is utilized for disinfecting and sterilizing the contaminated medical equipment through ultraviolet light irradiation.

According to another embodiment, the low-temperature sterilization device further comprises a photocatalyst agent. The photocatalyst agent such as TiO2 is employed into the reaction tank to facilitate photocatalytic reactions wherein the photocatalyst reacts with ultraviolet light, thereby enhanced the disinfecting and sterilizing of the contaminated medical equipment.

According to another embodiment, the low-temperature sterilization device further comprises an ozone generation device. Ozone produced by the ozone generation device is conveyed to the reaction tank through an aeration device to mix with the aqueous solution.

According to another embodiment, the low-temperature sterilization device further comprises a drying pump, for drying the medical instruments after disinfection and sterilization.

According to another embodiment, the chemical agents is one or more mixed disinfectants or sterilant, and the chemical agents may be selected at an appropriate concentration and formulation as needed.

According to an embodiment, a low-temperature sterilization method for placing contaminated medical equipment into a low-temperature sterilization device and using chemicals such as a slightly acidic electrolyzed water for disinfection and sterilization, comprises the following steps. First, one of a plurality of automated running programs on the operating unit is selected for disinfection and sterilization. Second, a metering unit is used to calculate a required dosage of the chemical agents based on the solution volume in the reaction tank to maintain the solution concentration within the range of 100 ppm to 10000 ppm, or ORP within the range of 800 mv to 1500 mv. Third, the following steps are performed simultaneously. An ultrasonic device is performed ultrasonic vibration according to the selected automated running program to detach adhered contaminants, bacteria and virus from the contaminated medical equipment. A feeding pump is used to control to feed the required dosage of the chemical agents into the reaction tank, an ozone generation device is used to introduce ozone into the reaction tank. An ultraviolet light device is used to disinfect and sterilize the contaminated medical equipment through ultraviolet lights. A recirculation pump is used to transport the aqueous solution with chemical agents to the medical equipment with tubing in the reaction tank to ensure contacting with the contaminated medical equipment for disinfection and sterilization reaction. Finally, the oversaturated ozone gas will rise to the top of the reaction tank and form a sterilized space with ultraviolet light.

According to another embodiment, the chemical agents is one or more mixed disinfectants, and the chemical agents may be selected at an appropriate concentration and formulation as needed.

According to another embodiment, the low-temperature sterilization device further comprises a drying pump, for drying the medical instruments after disinfection and sterilization.

According to an embodiment, a concentration control method for a low-temperature sterilization device, comprises the following steps. First, an operating device is used to set the chemicals concentration of the aqueous solution in a reaction tank. Second, a metering unit is used to calculate the required dosage of the chemical agents based on the volume of the solution in the reaction tank to maintain the concentration of the solution within the range of 100 ppm to 10000 ppm. Third, a feeding pump is used to import the required dosage of the chemical agents into the reaction tank. Fourth, an ozone generation device generates ozone. Finally, a recirculation pump is used to transport the chemical agents, aqueous solution, and the ozone into the medical equipment tubing or channels to ensure thorough mixing in the tank.

According to another embodiment, the recirculation pump is connected respectively to the channels of medical equipment and the reaction tank, for delivering a required dosage of chemical agents into the contaminated medical equipment channels.

According to another embodiment, the chemical agents is one or more mixed multiple disinfectants, and the chemical agents may be selected at an appropriate concentration and formulation as needed.

To sum up, the effect of the present invention includes: (1) Achieving efficient disinfection and sterilization of medical equipment through a combination of a plurality of methods including ultrasonic waves, chemical agents, ozone, ultraviolet light, and photocatalysts. (2) Employing a low-temperature sterilization method to avoid equipment damage or structural changes. (3) Real-time monitoring and adjustment of the solution concentration by detection units and metering units to ensure that the disinfectant remains within a certain range, thereby enhancing the stability and reliability of sterilization. (4) Using diluted disinfectants with low concentrations that are harmless to the environment and operators, allowing for direct discharge after use and eliminating the issue of secondary pollution. (5) Reducing the labor and time required and improving the quality for precleaning medical equipment, alleviating the operational burden on healthcare personnel, and reducing the quantity of spare parts needed for medical equipment. (6) Saving costs and manpower by employing automated control devices, thus reducing the need for manual operation. (7) Providing a sterile reaction space to ensure that both the gas phase and liquid phase in the reaction tank reach a sterile state, thereby avoiding secondary contamination of equipment during the disinfection process.

### BRIEF DESCRIPTION OF THE DRAWINGS

In order to make the above-described technology of this disclosure, as well as other aspects, features, advantages, and embodiments, more apparent and understandable, the description of the accompanying drawings is provided as follows.

FIG. 1 is a schematic diagram of a low-temperature disinfection and sterilization device of the prior art.

FIG. 2 is a front sectional view of a low-temperature sterilization device according to one embodiment of the present invention.

FIG. 3 is a three-dimensional schematic diagram of a low-temperature sterilization device according to one embodiment of the present invention.

FIG. 4 is a side sectional view of the low-temperature sterilization device according to one embodiment of the present invention.

### DETAILED DESCRIPTION

The detailed description provided below in connection with the appended drawings is intended as a description of the present examples and is not intended to represent the only forms in which the present example may be constructed or utilized. The description sets forth the functions of the example and the sequence of steps for constructing and operating the example. However, the same or equivalent functions and sequences may be accomplished by different examples.

To provide a more detailed explanation of various embodiments of the present invention, the following is accompanied by drawings for illustration. It should be understood that when an element is referred to as being “connected” or “disposed” on another element, it can indicate that the element is directly located on the other element, or there may be an intermediate element connecting the element to the other element. Conversely, when an element is described as being “directly on” or “directly connected to” another element, it is explicitly defined that there is no intermediate element involved.

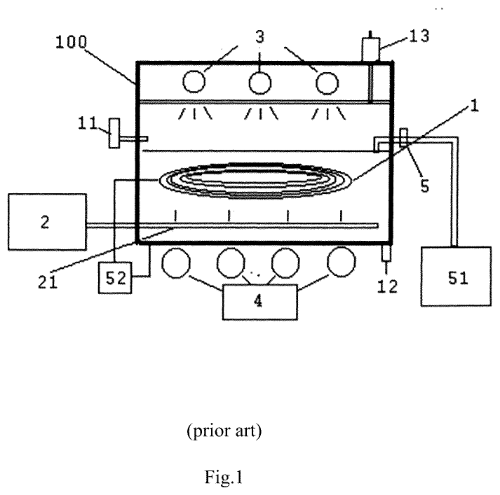

Referring to FIG. 1, description of related art provides a low-temperature disinfection and sterilization method along with its apparatus. This method primarily targets the disinfection, sterilization, and decontamination of contaminated reusable medical equipment. The process involves rapid detachment, destruction, and decomposition reactions using ultrasound, ultraviolet light, ozone, and disinfectants in aqueous solutions. This allows for deep and thorough cleaning, disinfection, and sterilization of various contaminated medical equipment, ensuring complete removal and eradication of contaminants, bacteria, and viruses. Furthermore, the applicant has made further improvements by proposing an innovative low-temperature sterilization device. This device adjusts the concentration of the aqueous solution through measurement and metering units to ensure the disinfectant concentration within a certain range. Additionally, it can automatically perform cleaning using ultrasound, enhancing sterilization stability and reliability. The invention can accomplish automatic cleaning and disinfection/sterilization of contaminated reusable medical equipment at room temperature in approximately 30 minutes, significantly reducing the labor and time required for cleaning such equipment.

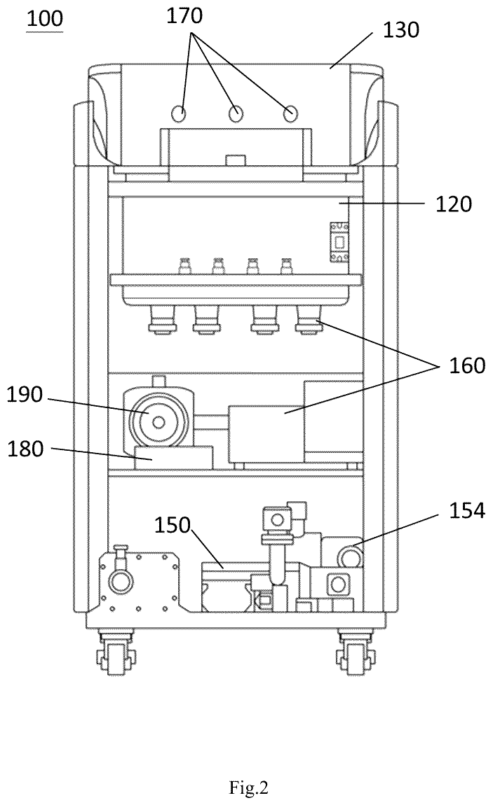

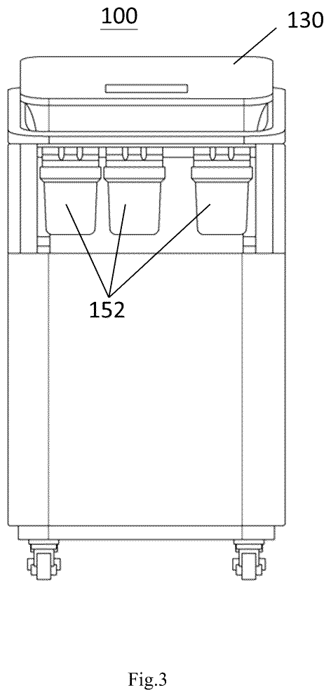

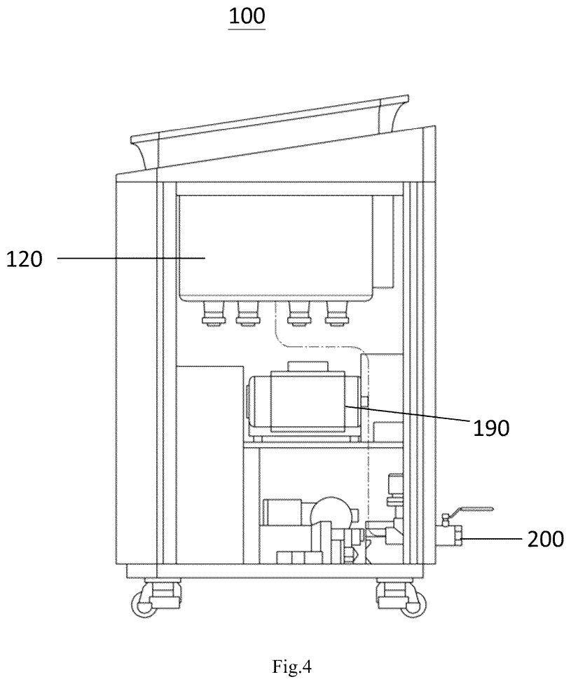

Referring to FIGS. 2 to 4, the present invention provides a low-temperature sterilization device 100 and method, particularly employing a combination of various techniques such as ultrasound, chemical agents, ozone, ultraviolet light, and photocatalysts to meet the requirements of FDA Guidance on Premarket Notification 510 (k), Submissions for Automated Endoscope Washers, Washer/Disinfectors, and Disinfectors Intended for Use in Health Care Facilities for achieving efficient disinfection and sterilization of medical equipment.

According to the notification issued by the Ministry of Health regarding the “Technical Specifications for Endoscope Cleaning and Disinfection Operations (2004 Edition),” the cleaning, disinfection, or sterilization of endoscopes and accessories must adhere to the following principles.
1. Endoscopes and accessories that enter sterile tissues or organs within the human body, or those that enter sterile body cavities through surgical incisions, such as laparoscopes, arthroscopes, brain mirrors, cystoscopes, cavity mirrors, etc., must be sterilized.
2. Endoscope accessories that penetrate mucous membranes, such as biopsy forceps, high-frequency electric knives, etc., must be sterilized.

The requirements of slightly acidic electrolyzed water for killing microorganisms are shown in Table 1.

TABLE 1
Disinfection time and removal efficiency of acidic
electrolyzed water for killing microorganisms

| | Disinfection of | | Disinfection of items that | |
|---|---|---|---|---|
| | cleaned items | | have not been cleaned | |
| | Disin- | Log of | Disin- | Log of |
| The target for | fection | reduction | fection | reduction |
| sterilization | time (min) | value | time (min) | value |
| Staphylococcus | ≤1.0 | ≥5.0 | ≤3.0 | ≥5.0 |
| aureus | | | | |
| ATCC 6538 | | | | |
| Escherichia | ≤1.0 | ≥5.0 | ≤3.0 | ≥5.0 |
| coli 8099 | | | | |
| Candida albicans | ≤1.0 | ≥4.0 | ≤3.0 | ≥5.0 |
| ATCC 10231 | | | | |
| Pseudomonas | ≤1.0 | ≥5.0 | ≤3.0 | ≥5.0 |
| aeruginosa | | | | |
| ATCC 15442 | | | | |
| Poliovirus Type | ≤3.0 | ≥4.0 | ≤5.0 | ≥5.0 |
| I vaccine strain | | | | |

Based on the above, the low-temperature sterilization device 100 uses slightly acidic electrolyzed water to disinfect and sterilize contaminated medical equipment, ensuring their safety and hygiene for reuse. Its main components include a reaction tank 120, an operating device 130, a metering unit 154, an ultrasonic device 160, a feeding pump, and a recirculation pump 150. It is required that the slightly acidic electrolyzed water used meets the standards specified in Table 1.

The reaction tank 120 is used to contain the contaminated medical equipment for disinfection and sterilization treatment. The design of the reaction tank 120 ensures that the equipment is fully immersed in the disinfectant solution to achieve thorough disinfection.

The operating unit 130 is positioned above the reaction tank 120 and provides a user interface for selecting one of a plurality of automated running programs to control and monitor the entire disinfection and sterilization process. The operating unit 130 may include control buttons, a touchscreen, or other interactive interfaces.

TABLE 2
Automated Running Programs

| Program | | | | Adjustment |
|---|---|---|---|---|
| Name | Process | Functions activated | time(s) | range (s) |
| F1 | Clean | Ultrasonic, | 180 | 0~9999 |
| Standard | | ultraviolet, ozone, | | |
| sterilization | | sterilizing agent | | |
| | Disinfect | Ultrasonic, | 180 | 0~9999 |
| | | ultraviolet, ozone, | | |
| | | sterilizing agent | | |
| | Sterilize | Ultrasonic, | 900 | 0~9999 |
| | | ultraviolet, ozone, | | |
| | | sterilizing agent | | |
| | Rinse | Sterile water, | 120 | 0~9999 |
| | | ultrasonic, | | |
| | | ultraviolet, ozone | | |
| F2 | Clean | Ultrasonic, | 180 | 0~9999 |
| Short range | | ultraviolet, ozone, | | |
| | | sterilizing agent | | |
| | Disinfect | Ultrasonic, | 180 | 0~9999 |
| | | ultraviolet, ozone, | | |
| | | sterilizing agent | | |
| | Sterilize | Ultrasonic, | 300 | 0~9999 |
| | | ultraviolet, ozone, | | |
| | | sterilizing agent | | |
| | Rinse | Sterile water, | 120 | 0~9999 |
| | | ultrasonic, | | |
| | | ultraviolet, ozone | | |
| F3 | Clean | Ultrasonic, | 180 | 0~9999 |
| Small volume | | ultraviolet, ozone, | | |
| equipment | | sterilizing agent | | |
| | Disinfect | Ultrasonic, | 180 | 0~9999 |
| | | ultraviolet, ozone, | | |
| | | sterilizing agent | | |
| | Sterilize | Ultrasonic, | 600 | 0~9999 |
| | | ultraviolet, ozone, | | |
| | | sterilizing agent | | |
| | Rinse | Sterile water, | 120 | 0~9999 |
| | | ultrasonic, | | |
| | | ultraviolet, ozone | | |
| F4 | Clean | Ultrasonic, | 180 | 0~9999 |
| Self- | | ultraviolet, ozone, | | |
| sterilization | | sterilizing agent | | |
| | Sterilize | Ultrasonic, | 600 | 0~9999 |
| | | ultraviolet, ozone, | | |
| | | sterilizing agent | | |
| | Rinse | Sterile water, | 120 | 0~9999 |
| | | ultrasonic, | | |
| | | ultraviolet, ozone | | |
| F5 | Dry | Air pump | 600 | 0~9999 |
| F6 | Leak test | Sterile water, Air | 180 | 0~9999 |
| | | pump | | |

The interface of the operating unit 130 also displays the various steps and their stages of the automated running program.

The metering unit 154 is positioned at the lowermost level inside the low-temperature sterilization device 100, and it calculates the required dosage of the chemical agents based on the solution volume in the reaction tank 120 to ensure that the concentration of the disinfectant remains within the range of 100 ppm to 10000 ppm or ORP within the range from 800 mv to 1500 mv.

The ultrasonic device 160 is positioned around the exterior of the reaction tank 120. When medical equipment is placed into the reaction tank 120 and the aqueous solution is injected, the ultrasonic device 160 generates ultrasonic vibrations. The ultrasonic device 160 generates ultrasonic vibrations to detach the adhered contaminants, bacteria, and viruses from the contaminated medical equipment. These vibrations, which are high-frequency oscillations, having enough energy to desorb contaminants from the surface of the equipment and further more disrupt the cell membrane structures of bacteria, viruses, and other microorganisms, leading to their death. Additionally, the ultrasonic vibrations create cavitation forces with tiny bubbles on the surface of the object. When these bubbles collapse, they release high-temperature and high-pressure energy, further damaging the cell structures of bacteria. Moreover, the ultrasonic vibrations create eddy currents and microstream, which flush or discharge dirt and microorganisms adhering to the surface or interior of the contaminated medical equipment, thus achieving the effect of cleaning and removing contaminants. In summary, the ultrasonic device 160 is an efficient, rapid, and hands-free automated cleaning method that effectively ensures the disinfection and cleanliness of medical instruments, thereby enhancing the safety and reliability of medical equipment.

The feeding pump installed within the operating unit 130, for importing the chemical agents from the chemical tank 152 into the reaction tank 120 according to the required dosage.

The recirculation pump 150 is connected respectively to the channels of medical equipment and the reaction tank 120. The recirculation pump 150 is responsible for delivering the chemical agents to the reaction tank 120, ensuring that the disinfectant in the solution fully covers and contacts the surface of the medical equipment, thereby achieving thorough disinfection/sterilization. The chemical agents, which can be a single or a combination of disinfectants, is released into the aqueous solution and works through chemical reactions to destroy and eliminate harmful microorganisms such as bacteria, viruses, and others on the surface and inside of the equipment, enhancing the disinfection effect. Examples of such chemical agents include super oxidants like hypochlorous acid or other chlorine-containing compounds used for microbial eradication purposes. Chlorine-containing compounds and similar chemical agents rapidly oxidize the cell membranes and cell walls of bacteria, viruses, fungi, and other microorganisms, leading to their demise. These chemical agents can effectively disinfect medical equipment at appropriate concentrations, while being relatively safe for the environment and human health, as they quickly break down into water and harmless salts. In low-temperature sterilization processes, hypochlorous acid or chlorine-containing compounds can serve as effective chemical disinfectants/sterilant. When used in conjunction with other disinfection methods, they can enhance the disinfection and sterilization effectiveness, ensuring the thorough cleanliness and safe reuse of medical equipment.

The low-temperature sterilization device 100 further comprises a drying pump 190. The drying pump 190 is located at the middle level inside the low-temperature sterilization device 100, and is used for drying the medical instruments after disinfection and sterilization.

According to one embodiment, the method for controlling the solution concentration in the low-temperature sterilization device 100 is implemented through the following steps.
1. The operating unit 130 is used to set the chemicals concentration of the solution in the reaction tank 120, adjusting the desired chemicals concentration of the disinfectant according to specific requirements.
2. The metering unit 154 calculates the required dosage of the chemical agents based on the volume of the solution in the reaction tank 120, to maintain the solution concentration in the range of 100 ppm to 10000 ppm. The volume range of the reaction tank is from 10 L to 100 L. For instance, if the volume of the solution in the reaction tank 120 is 10 L and the concentration of the disinfectant is set to 100 ppm, then the required dosage of the chemical agents is 1 milliliter.
3. The feeding pump imports the chemical agents from the chemical tank 152 into the reaction tank 120 according to the required dosage of the chemical agents.
4. The recirculation pump 150 is responsible for recirculating the aqueous solution with chemical agents diluted in the reaction tank 120 to contaminated medical equipment channels or tubing to ensure thorough mixing and contact. Simultaneously, it also transports the dissolved ozone water generated by the ozone generation device 180 along with the aqueous solution to contact with medical equipment in the reaction tank 120.

Through the above steps, the low-temperature sterilization device 100 can ensure that the concentration of disinfectant remains within the required range, thereby enhancing the stability and reliability of disinfection and sterilization.

In another embodiment, the low-temperature sterilization device 100 further comprises a UV light device 170, such as UV lamps, for disinfecting and sterilizing contaminated medical equipment through UV irradiation. UV light possesses strong germicidal properties, effectively disrupting the nucleic acids of bacteria and viruses, rendering them inactive. In the low-temperature sterilization device 100, the UV light device 170 is positioned on the back panel of the operating unit 130, above the reaction tank 120, ensuring that the medical equipment is adequately exposed to UV irradiation. Once the medical equipment is placed into the reaction tank 120, the UV light device 170 automatically activates, irradiating the equipment with UV light for a specified duration to ensure thorough disinfection and sterilization.

In another embodiment, the low-temperature sterilization device 100 may further comprises a photocatalyst device, which utilizes photocatalysis in conjunction with UV light to perform a photochemical reaction, disinfecting and sterilizing contaminated medical equipment. A photocatalyst is a special material with catalysts on its surface that can absorb light energy and initiate chemical reactions. Photocatalysts such as titanium dioxide (TiO2) being the most common and can be used in the reaction. When UV light shines on the surface of the photocatalyst, the catalysts on the photocatalyst react with water or oxygen in the air, generating reactive oxygen species (such as hydroxyl radicals), thereby oxidizing and decomposing organic pollutants and microorganisms.

In another embodiment, the low-temperature sterilization device 100 further comprises an ozone generation device 180, which is used to generate ozone and apply it to the disinfection and sterilization process. Ozone is a strong oxidant with powerful disinfectant and bactericidal properties. In the low-temperature sterilization device 100, the ozone generation device 180 typically generates ozone through specific methods and introduces it into the reaction tank 120 or onto the equipment, where it interacts with pollutants and bacteria, thereby achieving disinfection and sterilization.

The ozone generation device 180 typically employs technologies such as electrolytic ozone generators or ultraviolet ozone generators to produce ozone. An electrolytic ozone generator utilizes the electrolysis process to decompose oxygen into ozone and oxygen gas, while an ultraviolet ozone generator utilizes ultraviolet radiation to convert oxygen into ozone. The generated ozone is then transported to the reaction tank 120 or the equipment, where it undergoes oxidation reactions with pollutants and bacteria, achieving disinfection and sterilization. When ozone comes into contact with bacteria, viruses, and other microorganisms, it disrupts their cell membranes and nucleic acids, leading to their death. During the disinfection process, ozone rapidly decomposes into oxygen, leaving no harmful residues. Additionally, the oversaturated ozone gas rises to the top of the reaction tank 120 and forms a sterile space with ultraviolet light, preventing secondary contamination of the equipment during the disinfection process.

Various disinfection and sterilization methods were tested and evaluated in the laboratory, including but not limited to ultrasonic waves, chemical agents, ultraviolet light, and photocatalysis. Their effectiveness in disinfecting and sterilizing medical equipment and their ability to kill residual microorganisms were evaluated through experiments. These tests included the following steps.

1. Pre-processing of specimens: Firstly, the specimens were cleaned and disinfected to ensure their sterility.
2. Preparation of test bacterial liquid: The test bacterial liquid was prepared, including the activation of bacterial strains and the preparation of suspended bacterial liquid.
3. Inoculation of test bacterial liquid: According to the requirements of the test literature, the bacterial liquid was inoculated using a method that meets the culture conditions. Typically, a bacterial liquid concentration of 108 was used, with 1 milliliter placed in 100 milliliters of sterile water cup for testing.
4. Disinfection and sterilization of the above test groups were carried out using the method provided by the present invention.

### 4.1 Leakage Testing

During the F6 process of equipment operation, a leakage test was conducted by the low-temperature sterilization device 100, with the pressure set to 15 kPa.

### 4.2 Cleaning Procedure

The inner and outer surfaces of the endoscope were precleaned by the low-temperature sterilization device 100. The set time unit was 180 seconds. During this step, contaminants adhering to the instruments were dislodged using ultrasonic waves at a frequency of 40,000 cycles per second, effectively removing them from both the inner and outer surfaces of the instrument. Subsequently, the dislodged contaminants were decomposed and destroyed using ultraviolet light, ozone, and disinfectant. After the treatment process, the liquid will be discharged through the drain pip 200.

### 4.3 Sterilization Procedure

### 4.3.1 Selection and Requirements of Disinfectants/Sterilant

During this step, contaminants adhering to the instruments were detached using ultrasonic waves at a frequency of 40,000 cycles per second. Subsequently, the contaminants were decomposed and destroyed using ultraviolet light, ozone, and disinfectants. The selection of disinfectants must comply with the equipment manual and relevant health standards. The chosen disinfectant should meet the following requirements when the sterilization time specified in the sterilizer manual is 900 seconds.
(1) Capable of killing lg6 Escherichia coli and Staphylococcus aureus spores.
(2) Capable of killing lg6 Candida albicans, Bacillus subtilis, and Aspergillus niger.
(3) Capable of killing lg6 Bacillus anthracis var nigricans.

### 4.3.2 Sterilization Treatment

(1) The low-temperature sterilization device 100 has a calculating function. Disinfectants dosages are carefully calculated for one-time use only and must not be reused to ensure that the disinfectants maintain their microbial killing performance and consistent quality.
(2) Sterilization simulation experiments (as shown in Table 3) should demonstrate that under the standard sterilization time and concentration as described in the manual, all of the following requirements are met: removal lg6 of Escherichia coli, Staphylococcus aureus, Pseudomonas aeruginosa; removal of lg6 Candida albicans, Bacillus subtilis, and Aspergillus niger; removal of lg6 Bacillus subtilis var niger spores.
(3) The detection method for sterilization effectiveness complies with the requirements of the “Sterilization Technology Specification” (2002 edition).

### 4.4 the Final Rinse

The final rinse of both the inner and outer surfaces of the endoscope was conducted by the low-temperature sterilization device 100. The rinse is set in seconds, with a range of 0 to 9999 seconds. The water used for rinsing during this step is discharged after processing and cannot be reused.

### 4.5 The Drying Step

(1) The low-temperature sterilization device 100 has a drying procedure. During the drying step, 70% of alcohol along with filtered air are introduced into the inside the instrument's channels after cleaning and sterilization. After the automatic program is completed, the internal and external surfaces of the instruments should not contain excessive moisture and do not need to be wiped dry before use.
(2) The drying of both the inner and outer surfaces of the endoscope was performed by the low-temperature sterilization device 100. The drying is set in seconds, with a range of 0 to 9999 seconds.
(3) During the drying step, alcohol is flushed into the lumen of the channels. The duration of alcohol infusion is set in the range of 0 to 9999 seconds

### 5. Testing Step

The remaining bacterial count was tested.

### 6. Antimicrobial Efficacy Assessment

Antimicrobial efficacy was inferred from the residual bacterial count, as illustrated in Tables 3 and 4 below.

TABLE 3
presents the operational steps, durations, and clearance effectiveness
of the low-temperature sterilization device.

| | | | | clearance |
|---|---|---|---|---|
| Program | | | time | effectiveness |
| Name | Step | Subprocess | (s) | (%) |
| F1 Standard | Clean | Ultrasonic, | 180 | >90 |
| sterilization | | ultraviolet, ozone, | | |
| | | sterilizing agent | | |
| | disinfect | Ultrasonic, | 180 | >99.9 |
| | | ultraviolet, ozone, | | |
| | | sterilizing agent | | |
| | Sterilize | Ultrasonic, | 900 | >99.9999 |
| | | ultraviolet, ozone, | | |
| | | sterilizing agent | | |

TABLE 4
shows the indicator bacteria, time, and clearance
effectiveness of the standard sterilization process
run by the low-temperature sterilization device

| | | | | clearance |
|---|---|---|---|---|
| Program | | | time | effectiveness |
| Name | Step | Indicator bacteria | (s) | (%) |
| F1 Standard | Clean | Escherichia coli | 180 | lg6 |
| sterilization | disinfect | Staphylococcus aureus, | 180 | lg6 |
| | | Pseudomonas | | |
| | | aeruginosa, | | |
| | | Candida albicans, | | |
| | | Bacillus subtilis, | | |
| | | Aspergillus niger | | |
| | Sterilize | Bacillus subtilis | 900 | lg6 |
| | | var niger spores | | |

Based on Tables 3 and 4, it may be observed that this invention achieves close to 100% sterilization. Hypochlorous acid (HOCl) is the primary active ingredient in the disinfectants/steriliants, with effective chlorine concentrations ranging from 10 mg/L to 100 mg/L. Toxicological risk assessment tests have confirmed that the waste liquid generated after the reaction is safe and non-toxic. It can be directly discharged into the hospital's sewage collection system without further treatment, thus comparing to other traditional chemical disinfections methods used, this is a significant saving on treatment costs.

In summary, this invention has several advantages. Firstly, by employing a combination of methods such as ultrasonic waves, chemical agents, ozone, ultraviolet light, and disinfectants, it efficiently achieves the sterilization and disinfection of medical equipment without risking damage or structural changes to the devices. Real-time monitoring and adjustment of the concentration of the disinfectant ensure its stability and reliability within a certain range. The disinfectants used are harmless at low concentrations after dilution, posing no risk to the environment or operators, and can be directly discharged after use, thus avoiding secondary pollution issues. Additionally, this invention reduces the labor and time required for precleaning medical equipment, improves the quality, alleviates the workload of healthcare personnel, reduces the need for spare medical equipment, and consequently saves costs and labor associated with manual operations.

The present invention is disclosed herein with reference to preferred embodiments, but it will be understood by those skilled in the art that the above embodiments are provided for descriptive purposes and not intended to limit the scope of the patent claims of the present invention. Any changes or substitutions equivalent to the above embodiments should be interpreted as being within the spirit or scope of the present invention. Therefore, the scope of protection of the present invention should be determined by the claims set forth above.

What is claimed is:

1. A low-temperature sterilization device utilized slightly acidic electrolyzed water to disinfect and sterilize a contaminated medical equipment placed within the device, effectively removing adhered contaminants, bacteria and virus from all crevices and corners of the contaminated medical equipment, wherein the low-temperature sterilization device comprising:
a reaction tank arranged for accommodating the contaminated medical equipment to proceed with disinfection and sterilization process;
an operating device positioned above the reaction tank, for providing users to optionally select one of a plurality of automated running programs to control and monitor all the disinfection and sterilization process;
an ultrasonic device utilized to detach the adhered contaminants, bacteria and virus from the contaminated medical equipment through ultrasonic vibrations with the frequency ranges from 20 hertz to 1 giga hertz;
a metering unit for calculating the required dosage of the chemical agents based on the volume of the aqueous solution in the reaction tank, to provide a concentration of the aqueous solution within a range of 100 ppm to 10000 ppm;
a feeding pump installed within the operating unit, for importing the chemical agents from the chemical tank into the reaction tank according to the required dosage;
a recirculation pump connected respectively to the channels of medical equipment and the reaction tank, for delivering a required dosage of chemical agents into the contaminated medical equipment channels.

2. The low-temperature sterilization device of claim 1, further comprising an ultraviolet (UV) light device, utilized for disinfecting and sterilizing the contaminated medical equipment through ultraviolet light irradiation.

3. The low-temperature sterilization device of claim 1, further comprising a photocatalyst device, employed to facilitate photocatalytic reactions wherein a photocatalyst reacts with ultraviolet light, thereby disinfecting and sterilizing the contaminated medical equipment.

4. The low-temperature sterilization device of claim 1, further comprising an ozone generation device, wherein ozone produced by the ozone generation device is conveyed to the reaction tank via an aeration tube to combine with the aqueous solution.

5. The low-temperature sterilization device of claim 1, further comprising a drying pump, for drying the medical instruments after disinfection and sterilization.

6. The low-temperature sterilization device of claim 1, wherein the chemical agents is one or more mixed multiple disinfectants, and the chemical agents is selected from an appropriate concentration and formulation as needed.

7. A low-temperature sterilization method for placing a contaminated medical equipment into a low-temperature sterilization device and using slightly acidic electrolyzed water for disinfection and sterilization, comprising:
selecting one of a plurality of automated running programs from an operating unit for disinfection and sterilization;
calculating a required dosage of chemical agents from a metering unit based on a solution volume in a reaction tank to maintain a solution concentration within a range of 100 ppm to 10000 ppm or with ORP within the range of 800 mv to 1500 mv;
detaching adhered contaminants, bacteria and virus by performing an ultrasonic vibration of the contaminated medical equipment by a ultrasonic device according to the automated running program, releasing the required dosage of the chemical agents form a feeding pump controlled the chemical tank, generating ozone released by an ozone generation device, disinfecting and sterilizing the contaminated medical equipment through ultraviolet light from a ultraviolet light device, and transporting the solution and chemical agents to the reaction tank to contact the contaminated medical equipment from a recirculation pump for disinfection and sterilization reaction; and
rising oversaturated ozone gas to the top of the reaction tank and form a sterile space with ultraviolet light.

8. The low-temperature sterilization method of claim 7, wherein the chemical agents is one or more mixed multiple disinfectants, and the chemical agents may be selected at an appropriate concentration and formulation as needed.

9. The low-temperature sterilization method of claim 7, further comprising a drying pump, for drying the medical instruments after disinfection and sterilization.

10. A concentration control method for a low-temperature sterilization device, comprising:
setting the chemicals concentration of the aqueous solution in a reaction tank from an operating device;
calculating the required dosage of chemical agents from a metering unit based on the volume of the solution in the reaction tank to maintain the concentration of the solution within the range of 100 ppm to 10000 ppm;
importing the required dosage of the chemical agents from the chemical tank into a reaction tank by a feeding pump; and
generating ozone by an ozone generation device through the aqueous solution from a recirculation pump into the reaction tank.

11. The concentration control method for the low-temperature sterilization device of claim 10, wherein the recirculation pump connected respectively to the channels of medical equipment and the reaction tank, for delivering a required dosage of chemical agents into the contaminated medical equipment channels.

12. The concentration control method for the low-temperature sterilization device of claim 10, wherein the chemical agents is one or more mixed multiple disinfectants, and the chemical agents may be selected at an appropriate concentration and formulation as needed.
